(19) United States
(12) Patent Application Publication
(10) Pub. No.: US 2022/0198650 A1
(43) Pub. Date: Jun. 23, 2022

(54) METHOD AND APPARATUS FOR EFFICIENT MULTI-RESOLUTION IMAGE PROCESSING FOR OBJECT IDENTIFICATION AND CLASSIFICATION

(71) Applicant: Tarushii N Goel, McLean, VA (US)

(72) Inventor: Tarushii N Goel, McLean, VA (US)

(73) Assignee: Tarushii N Goel, McLean, VA (US)

(21) Appl. No.: 17/127,986

(22) Filed: Dec. 18, 2020

(51) Int. Cl. G06T 7/00; G06T 3/40; G06T 7/70; G06T 7/62; G06K 9/62; A61B 6/03; A61B 6/00

(52) U.S. Cl. CPC G06T 7/0012; G06T 2207/10016; G06T 7/70; G06T 7/62; G06K 9/6267; A61B 6/032; A61B 6/501; A61B 6/504; A61B 6/502; A61B 6/5252; G06T 2207/30068; G06T 2207/30096; G06T 2207/30016; G06T 2207/10081; G06K 2209/051; G06T 3/40

(57) ABSTRACT

This invention presents a system that can be used for object identification and classification by training multiple neural networks on large quantities of images efficiently, The first in a series of convolutional neural networks is trained on a low resolution version of the image; in each successive stage of the series a model is trained on a smaller and more specific subregion of the original image. GradCAM is used to identify an area of focus in the image which later models will classify. The models are strung together into a single mega-classifier. The training time of this approach is significantly less, as smaller and lower resolution images are easier to manipulate and the implementation of GradCAM presented is much faster than standard library implementations The effectiveness of the proposed approach is demonstrated by applying it on the task of Intracranial Hemorrhage detection and classification.

Intracranial hemorrhage is a critical brain injury characterized by bleeding and swelling in the tissue surrounding a broken artery. Hemorrhages often cause strokes which are the 5th leading cause of death in the U.S. Current diagnostic procedures need a highly trained radiologist with specialized training in identifying brain hemorrhage. As a result, diagnosis is expensive, and in remote areas where radiologists are hard to find, diagnosis is difficult and often inaccurate. My research develops a computer aid to radiologists that can screen brain scans to cut costs and accelerate diagnosis. Through image windowing, data augmentation, and Convolutional Neural Networks (CNNs), the system I present achieves high accuracies in detecting hemorrhage and 5-way subtype classification. The system consists of a two-model ensemble; one model is trained to detect hemorrhage and potential regions of hemorrhage in the CT scans, and the second model analyzes hemorrhagic regions found by the first model more closely. The two-model ensemble reduces the error rate by 17% relative to the first model alone, increasing the overall detection accuracy to 97.0%. It also applies Gradient Class Activation Maps (GradCAM), which provide a coarse mapping of the regions of the image that were most influential in the model's predictions. The activation maps provide a strong visual aid for explaining and justifying the model's outputs and can be used by radiologists to assist them in identifying the areas of focus in an image.

### Description

Devices that search and identify objects need to process large amounts of data using a computing device. If the images are processed at lower resolution then the precision and recall accuracies may degrade. If very high resolution images are used, the amount of data processing may explode. This invention presents a technique to build a device that will perform image processing for object search and identification in a multi-resolution manner, to control the computational cost, while still maintaining high accuracies. In the field of deep Neural Networks, models continue to become deeper the corresponding datasets for training the models become larger.Particularly, large quantities of high-resolution images paired with the millions of parameters in state-of-the-art computer vision models, totals to an exceptionally long training process. If the image is very high resolution, this would also limit the batch size and therefore the quality of training. A simple solution is to lower the resolution of the images, but this sacrifices minute, yet potentially important, details in images. Another, equally sacrificial solution, is to crop the image, which preserves the high resolution but forgoes entire discarded regions of the image. This invention presents a combination of these two techniques, that uses multiple models and Gradient Class Activation Maps (GradCAM) to crop the images in a way that retains the most information. This solution improves the accuracy of classification while maintaining a reasonable training time.

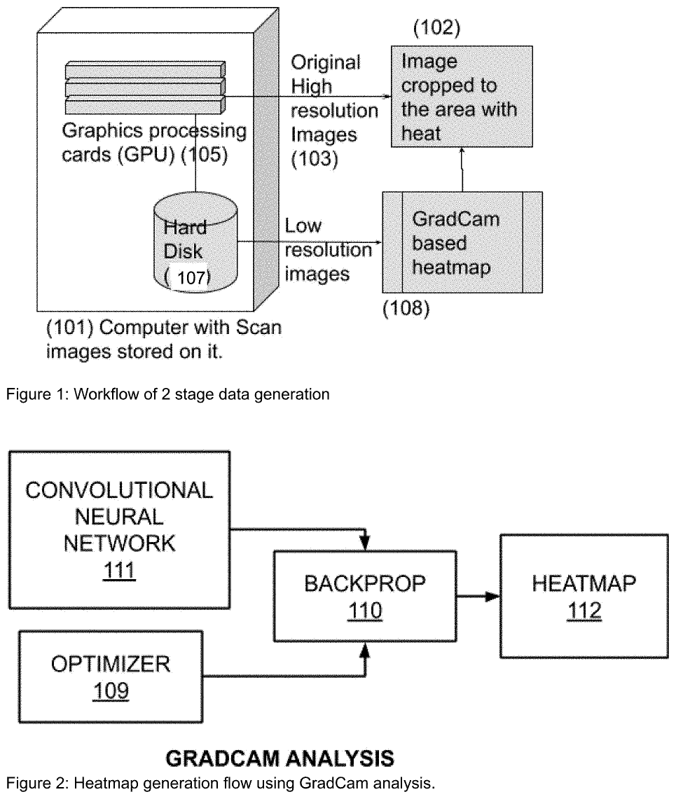

FIG. 1 shows the apparatus of the setup. The high resolution images(103) are stored on the hard disk(107) of a computer (101) along with their classification labels. The computer is equipped with a multiplicity of Graphics Processing Units (GPUs) (105). It's not practical to train a classification model with the high resolution images,

In the preferred embodiment of this invention, an image processing library such as OpenCV is used to subsample the images to a lower resolution such as 331×331. The subsampled images (106) are stored separately on the hard disk of the computer. The low resolution labels and the corresponding classification labels are used to train a convolutional neural network based classifier such as NasNet or Xception [1, 7]. Other architectures are also a possibility.

Gradient Class Activation Map (GradCAM) is a known technique used to explain the decision of a convolutional neural network [4]. It works on the output of the final and pre-final layers of a convolutional neural network, to create a heatmap indicating the region of interest that is responsible for the classification decision. The implementation of GradCAM in this invention contains optimizations in the code that cause it to run very fast, even on large datasets.

GradCam is applied to the classification output of the low resolution image to obtain a heat-map [4]. A centroid of that heatmap is calculated, and then the high resolution image is cropped in such a way that the centroid of the cropped image coincides with the centroid of the heat-map as shown in block 102 of FIG. 1.

The resulting high resolution but cropped image is then used as a new input to train a new neural network for the final classification of the image. Our implementation on GradCAM is novel and performs at a higher speed than many other implementations. An example code describing the implementation is shown below.

The Initialization portion is run only once in the beginning. GradCam approach (Depicted in FIG. 2) involves first getting the pooled backpropagation weights (110) for each of the class activation maps [4]. This requires the optimizer (109) to be initialized so that the back-propagation can be performed on the neural network (111) to obtain the heatmap (112). In our Keras based implementation, whereas the relevant portions of the code written in Python3 are shown below, that step is implemented only once in the method visualize-cam-init. The visualize-cam-run then takes the back-prop based optimizer and the image as input and applies GradCam, leading to significant time savings.

```
def visualize_cam_init
(self, layer_idx, penultimater_layer_idx, filter_indices=0):
  penultimate_layer = self.model.layers[penultimater_layer_idx]
  losses = [
    (ActivationMaximization
    (self.model.layers[layer_idx], filter_indices), −1)
  ]
  penultimate_output = penultimate_layer.output
  opt = Optimizer(self.model.input, losses, wrt_tensor=penultimate_output,
norm_grads=False)
  return opt
def visualize_cam_run
(self,layer_idx, penultimater_layer_idx, opt, seed_input):
  input_tensor = self.model.input
  penultimate_layer = self.model.layers[penultimater_layer_idx]
  penultimate_output = penultimate_layer.output
  _, grads, penultimate_output_value =
  opt.minimize(seed_input, max_iter=1,
grad_modifier=None, verbose=False)
  #opt.minimize(seed_input, max_iter=1, grad_modifier=grad_modifier,
verbose=False)
  # For numerical stability. Very small grad values along with small
penultimate_output_value can cause
  # w * penultimate_output_value to zero out,
  even for reasonable fp precision of
float32.
  grads = grads / (np.max(grads) + K.epsilon( ))
  # Average pooling across all feature maps
  # This captures the importance of feature map
  (channel) idx to the output.
  channel_idx = 1 if K.image_data_format( ) = = ‘channels_first’ else −1
###############sus
  other_axis = np.delete(np.arange(len(grads.shape)), channel_idx)
  weights = np.mean(grads, axis=tuple(other_axis))
  # Generate heatmap by computing weight * output over feature maps
  output_dims = utils.get_img_shape(penultimate_output)[2:]
  heatmap = np.zeros(shape=output_dims, dtype=K.floatx( ))
  for i, w in enumerate(weights):
    if channel_idx = = −1:
      heatmap += w * penultimate_output_value[0, . . . , i]
    else:
      heatmap += w * penultimate_output_value[0, i, . . . ]
  #ReLU thresholding to exclude pattern mismatch information (negative
gradients).
  heatmap = np.maximum(heatmap, 0)
  # The penultimate feature map size is
  definitely smaller than the input image.
  heatmap = cv2.resize(heatmap, self.input_dims[:2], interpolation =
cv2.INTER_CUBIC)
  # Normalize and create heatmap.
  heatmap = utils.normalize(heatmap)
  return heatmap
  #return np.uint8(cm.jet(heatmap)[. . . , :3] * 255)
def Get_Activation_map(self, img, layer_idx, penultimater_layer_idx):
  if not self.isGradCamInit:
    self.opt = self.visualize_cam_init(layer_idx, penultimater_layer_idx)
    self.isGradCamInit = True
  return self.visualize_cam_run(layer_idx, penultimater_layer_idx, self.opt,
seed_input=img)
```

This approach allows us to train on millions of images without consuming exorbitant amounts of time and compute costs. It is often the case that GradCAM heatmaps are completely black, and no clear centroid exists. Therefore we add a small heat bias in the center of the image in the heatmap. This ensures that a centroid is found.

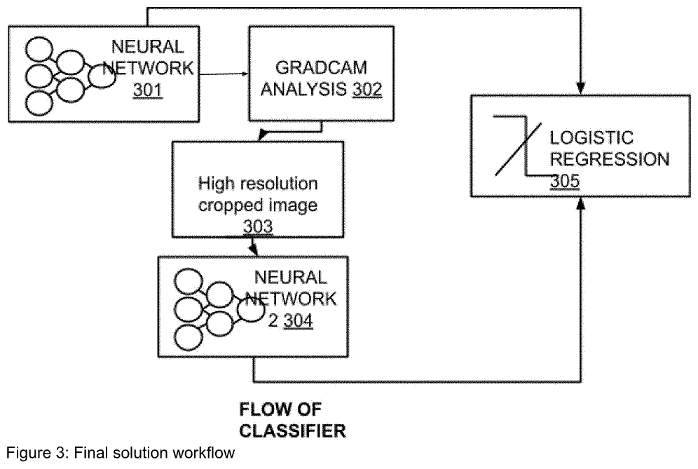

FIG. 3 shows the workflow of the final implemented solution. The initial neural network 301 analyzes low resolution images to identify the approximate area of interest. However since we use GradCam for localization, only class labels are needed for training this neural network and the location information is not required. Based on the region localized by the GradCam analysis (302) of the localized region is used to crop a section of the original high resolution image (303). A separate neural network (304) analyzes the high resolution image to classify and identify one more time. The results of neural network (301) and the neural network (304) are combined using logistic regression so that the final performance is maximized.

To test the usability and performance of this invention, CT scans of the brain are used, some normal, and others showing from five different types of intracranial brain hemorrhage, to train a deep CNN architecture to classify intracranial hemorrhage . The accuracy results were calculated for both single stage and two-stage multi-resolution approach.

Single-Stage:

| | 1—Equal Error | Precision-Recall |
|---|---|---|
| Type of Hemorrhage | Rate (Accuracy) | AUC |
| Any | 96.4% | 0.936 |
| Epidural | 99.0% | 0.753 |
| Intraparenchymal | 90.9% | 0.908 |
| Intraventricular | 94.9% | 0.911 |
| Subarachnoid | 88.3% | 0.790 |
| Subdural | 88.4% | 0.863 |

Two-Stage:

| | 1—Equal Error | Precision-Recall |
|---|---|---|
| Type of Hemorrhage | Rate (Accuracy) | AUC |
| Any | 97.0% | 0.947 |
| Epidural | 99.1% | 0.763 |
| Intraparenchymal | 91.4% | 0.908 |
| Intraventricular | 95.3% | 0.916 |
| Subarachnoid | 88.5% | 0.797 |
| Subdural | 88.7% | 0.865 |

References

[1] Chollet, F. (2017). Xception: deep learning with depthwise separable convolutions. 2017 IEEE Conference on Computer Vision and Pattern Recognition (CVPR), 1800-1807.

[2] Hssayeni, M. D., Croock, M. S., Al-Ani, A., Al-khafaji, H. F., Yahya, Z. A., Ghoraani, B. (2019). Intracranial hemorrhage segmentation using a deep convolutional model. doi:10.13026/w8q8-ky94

[3] Kuoa, W., Hanea, C., Mukherjeeb, P., Malika, J., Yuhb, E. L. (2019). Expert-level detection of acute intracranial hemorrhage on head computed tomography using deep learning. PNAS, 166(45), 22737-22745. doi:10.1073/pnas.1908021116

[4] Selvaraju, R. R., Das A., Vedantam R., Cogswell M., Parikh D., Batra D. (2016). Grad-CAM: why did you say that? Visual explanations from deep networks via gradient-based localization. International Journal of Computer Vision, 128(2), 336-359

[5] Shen, J., Zhang, C., Jiang, B., Chen, J., Song, J., Liu, Z., . . . Ming, W. K. (2019). Artificial intelligence versus clinicians in disease diagnosis: Systematic review. JMIR medical informatics, 7(3). doi:10.2196/10010

[6] Ye, H., Gao, F., Yin, Y., Guo, D., Zhao, P., Lu, Y., . . . Xia, J. (2019). Precise diagnosis of intracranial hemorrhage and subtypes using a three-dimensional joint convolutional and recurrent neural network. European Radiology, 29, 6191-6201. doi:10.1007/s00330-019-06163-2

[7] Zoph, B., & Vasudevan, V., Shlens, J., Le, Quoc. (2018). Learning transferable architectures for scalable image recognition. IEEE/CVF Conference on Computer Vision and Pattern Recognition, 8697-8710.. doi:10.1109/CVPR.2018.00907.

What is claimed is:

1. A device for search and classification of plurality of objects wherein the device is configured to
a. Access either live or previously captured Images or videos
b. Identify plurality of objects such as but not limited to:
b.i. Presence of Intracranial brain hemorrhage and its type in a brain CT scan
b.ii. Cancerous tumor in a breast scan
b.iii. Everyday objects such as tables and chairs in natural images
c. Process the images in a multi-resolution manner where initially a low resolution image is processed and then subsequently a zoomed in higher resolution image is processed for the area of interest.
d. Where GradCam technique is used to identify the area to zoom into for high resolution image processing.
e. Wherein the device provides information about the approximate location, size and identity of the object detected.

2. The device in claim 1, wherein the device is further configured to break down the GradCam computations into two parts as follows:
a. The first part is the initialization of the back-propagation parameter for the penultimate layer of the neural network. This part is executed only once at the time of device initialization.
b. The second part is to perform the remainder of the computation of GradCam steps involving the computation of neuron importance weights and the weighted aggregation of activation maps followed by ReLU activation.

3. The device in claim 2, wherein the device skips the step of high-resolution image processing, if the object is not found within a certain level of confidence.

4. The device in claim 3, wherein the device is used to identify and classify intracranial brain hemorrhage.
